(19) United States
(12) Patent Application Publication
(10) Pub. No.: US 2010/0196893 A1
(43) Pub. Date: Aug. 5, 2010

(54) Method for genotyping DNA tandem repeat sequences

(75) Inventors: Uwe R. Müller, Alachua, FL (US); Robert E. Wagner, JR., Nunn, CO (US)

Correspondence Address: THE MCCALLUM LAW FIRM, P. C., 685 BRIGGS STREET, PO BOX 929, ERIE, CO, 80516, US

(73) Assignee: Gene Check, Inc., Fort Collins, CO (US)

(21) Appl. No.: 12/589,022

(22) Filed: Oct. 15, 2009

Related U.S. Application Data
(60) Provisional application No. 61/197,286, filed on Oct. 24, 2008.

(51) Int. Cl. C12Q 1/68

(52) U.S. Cl. 435/6

(57) ABSTRACT

The present invention provides methods for determining the number of tandem repeat units in a region of double stranded DNA based on the use of RecA-like recombinase protein and oligonucleotide ligation. The methods of the present invention provide RecA coated, specific DNA oligonucleotide probes (RecA filaments) for homology searching in duplex DNA where the location of homologous sequences results in the formation of D-loop structures containing a duplex region comprising the oligonucleotide probe and one strand of the target DNA. The present invention further provides sets of oligonucleotide probes (ligation partners) selected to have sequence complementary to non-repeat sequence flanking a region of tandem repeats and sequence complementary to varying numbers of repeat units such that only that pair of oligonucleotides that can be aligned, via RecA mediated homology searching, with the target sequence such that their terminal bases are paired with adjacent nucleotides in the target sequence will be substrates for ligation. Thus, the present invention provides methods whereby successful ligation is diagnostic of the number of repeat units in the target DNA sequence. Also disclosed are compositions and kits useful for practicing the foregoing methods.

### 1. FIELD OF THE INVENTION

The present invention relates to the fields of molecular biology and medicine, specifically to methods for determining the number of tandem repeat units in sequences in double-stranded DNA.

### 2. DESCRIPTION OF THE BACKGROUND ART

### Small Tandem Repeat (STR) Sequences

Tandem repeat sequences are found in the genomes of all higher eukaryotes. Upon local melting, regions of tandem repeat sequences are frequently able to form relatively stable secondary structures (e.g. cruciforms and slippage structures). Such structures may serve as targets for nucleases or other DNA specific enzymes. Regions of tandem repeats generally exhibit substantial genetic instability, leading to heritable polymorphisms, genetic predisposition to disease, and disease itself. One class of STRs, the microsatellites, contains repeat units of 1-4 bases and occurs in human DNA approximately once every 20,000 nucleotides. The highly polymorphic nature of microsatellites makes them excellent markers for genetic mapping of specific loci or for differentiation between individual genomes. In addition, they are used in medical applications, such as the identification of fetal cells in maternal blood, or the monitoring of allogeneic bone marrow transplants.

A select group of STRs with a base repeat unit of 4 nucleotides is used for forensic identification. In 1997, the FBI announced the selection of 13 STR loci to constitute the core of the United States national database, the Combined DNA Index System or CODIS. Since then, three additional STRs have been added to the CODIS STRs and various commercial kits have been developed for STR genotyping. Depending on the number of STRs used, the discrimination power of the kits can range between 1:410 (3 STRs; Promega, released 1993) and 1:1.8×10¹⁷ (16 STRs; Promega PowerPlex 2.1). The CODIS system has also been adopted in many other countries, albeit with small variations in the number and type of STRs to match their needs based on the size and ethnic composition of their population.

Since a multiplex polymerase chain reaction (PCR) is used to amplify the chromosomal regions of interest, STR profiles can be determined with very small amounts of DNA. However, determination of the exact number of repeat units for each STR in the resulting PCR products requires their separation and accurate sizing. Traditionally this has been achieved by slab gel electrophoresis or capillary electrophoresis. Capillary electrophoresis can be performed in microfabricated channels or capillary arrays. More recently methods utilizing mass spectrometry and microarray technology have been developed in an attempt to reduce the time for analysis and/or increase throughput.

The availability of suitable technology for rapid, low-cost DNA typing will have a significant impact in the way DNA typing can be used in everyday police investigations, and will have widespread application outside the forensic arena. There are currently no automated systems available for DNA typing, and although robotics exist for sample extraction, PCR amplification and fragment analysis, the complete process requires user intervention and significant technical skill. Moreover, the traditional gel electrophoresis methodologies used for determining the size of the STR repeat are time consuming and require relatively large and complex pieces of equipment. Microfluidics-based systems have so far not reached the market place, possibly due to reliability issues, and mass spectrometry requires significant operator training and sophisticated and expensive instrumentation.

Microarray technology may be capable of displacing these technologies, since microarrays have been successfully implemented in a variety of DNA-based diagnostic applications, assay and analysis automation is practical and available, and the format is cost competitive. In principle, all STR loci could be analyzed on a single array, and by utilizing the target amplification and complexity reduction afforded by PCR, hybridization reactions could be relatively fast. However, determining the repeat length accurately by hybridization is technically very challenging and not possible with traditional probe designs and standard isothermal hybridization protocols. Efforts to overcome these issues have been described and have employed either enzymatic treatments of the hybridized DNA, multiple hybridization steps and neural network classification or the combination of two hybridization probes and electronic stringency manipulation to select for perfectly base-stacked probes. While the first two appear unsuitable for incorporation into an assay system that can address the above described market need, the last has been shown to suffer from lack of sufficient specificity.

Recombinase Mediated Ligation (RML) (Wagner U.S. Pat. No. 7,244,562) is a novel method of SNP and specific sequence detection and repeat sequence genotyping utilizing the highly specific homology searching ability of the bacterial recombination protein, RecA.

### Oligonucleotide Ligation Assays (OLA)

The OLA method of SNP detection utilizes two oligonucleotide probes which are complementary to the target DNA flanking the SNP/mutation site. One oligonucleotide ends at the SNP site and the other ends at the nucleotide adjacent to the SNP site such that, when annealed to target DNA, the ends of both oligonucleotides are base-paired at the site of the SNP and can be joined (ligated) by DNA ligase. If there is a mismatch at the joint, i.e., if the end base in the oligonucleotide that covers the SNP/mutation is not complementary to the SNP/mutation, ligation cannot occur. Ligated products can be detected on gels without labeling by virtue of their increased length relative to the oligonucleotide probes.

To avoid the use of gels, OLA has been performed with probes which allow ligation product immobilization. For example, one oligonucleotide probe can be prepared with a 5′ biotin adduct to allow immobilization to avidin- or streptavidin-coated plates or microspheres. This probe generally ends one nucleotide 5′ of the SNP. The other probe is detectably labeled, either internally or at the 3′ end. Detectable labels include fluorescent, radioactive and antigenic labels such as digoxigenin. In this example, the 5′ nucleotide of the detectably labeled probe would be complementary to one allele of the SNP or mutation. Target DNA must be denatured and the ligation oligonucleotides allowed to anneal. If the allele in question is present in the target DNA, ligation will occur and the labeled oligonucleotide will be linked to the biotinylated oligonucleotide such that label can be bound to avidin/streptavidin coated plates or microspheres. By combining two differently labeled oligonucleotides, each with their 5′ nucleotide complementary to a different allele of the SNP or mutation, it is possible to perform complete genotyping in a single assay.

OLAs have also been used to detect changes in mononucleotide repeat sequence lengths. The system is able to discriminate repeat sequences that vary by one nucleotide in sequences of up to 16 mononucleotide repeats. Notably, the above authors stated that: “The greatest source of error for analysis of mononucleotide repeat sequences, however, is the error generated during PCR amplification of microsatellite repeats.” OLA has been used with flow cytometry and limited multiplexing. In this system, ligation products were immobilized to microspheres (Luminex) by means of a 25 base specific oligonucleotide “tail”. Target DNA was PCR-amplified prior to ligation. Simultaneous genotyping of 9 SNPs was demonstrated.

By virtue of their precision and specificity OLAs have enjoyed widespread application in mutation and SNP detection. However, and in clear distinction to the present invention, OLAs absolutely require denaturation of target DNA. In addition, OLAs have not been successful without amplification of target sequences.

### RecA

RecA is the best characterized recombinase. RecA is a bacterial protein involved in DNA repair and genetic recombination and has been best characterized in E. coli. RecA is the key player in the process of genetic recombination, in particular in the search and recognition of sequence homology and the initial strand exchange process. RecA can catalyze strand exchange in the test tube. Recombination is initiated when multiple RecA molecules coat a stretch of single-stranded DNA (ssDNA) to form what is known as a RecA filament. This filament, in the presence of ATP, searches for homologous sequences in double-stranded DNA (dsDNA). When homology is located, a three stranded (D-loop) structure is formed wherein the RecA filament DNA is paired with the complementary strand of the duplex.

RecA homology searching is extremely precise and RecA has been used to facilitate screening of plasmid libraries for plasmids containing specific sequences. In this approach, biotinylated ssDNA probes are reacted with RecA to form RecA filaments. The filaments are used for homology searching in circular plasmid DNA. When the probes are removed by binding to avidin, those plasmids containing sequences homologous to the probes are isolated by virtue of the triple stranded (D-loop) structures formed by the RecA filament and the plasmid duplex. Stabilization of these structures requires the use of adenosine 5′-[.gamma.-thio]triphosphate (ATP[.gamma.-S]) in place of ATP. ATP[.gamma.—S] allows homology searching by RecA, but is non-hydrolyzable and thus does not allow RecA to dissociate from the triple stranded structure.

RecA has also been used, in a variety of applications, to facilitate the mapping and/or isolation of specific DNA regions from bacterial and human genomic DNA. In one of these applications, RecA is used in conjunction with restriction enzymes (sequence-specific double strand DNA endonucleases) to allow isolation or identification of specific DNA fragments. RecA filaments are prepared and reacted with genomic DNA under conditions that allow triple strand (D-loop) structure formation. The DNA is then treated with either a restriction endonuclease or a modification methylase (methylase action transfers a methyl group to the specific recognition sequence of a specific restriction endonuclease, thus protecting the sequence from endonuclease digestion). The presence of the RecA filament in the triple strand structure prevents digestion or methylation.

In a more recently developed application, specific RecA filaments were used to protect restriction endonuclease generated “sticky ends” from being filled in by DNA polymerase such that, upon removal of the RecA filaments, specific fragments can be cloned into plasmid vectors. In this application, genomic DNA is digested with one or more restriction enzymes that produce recessed 3′ ends. A specific fragment from this digestion is protected by triple strand structure formation with a pair of RecA filaments. The recessed 3′ ends of the remaining fragments are then filled in with a polymerase. The polymerase is removed or inactivated, the RecA filament is removed and the specific fragment cloned by virtue of its sticky ends.

RecA has been used in association with DNA ligase to label specific DNA fragments. Oligonucleotides are designed to allow the 3′ end to form a double-stranded region by folding back on a portion of itself (hairpin), RecA is then used to coat the remaining single-stranded 3′ region and the resulting RecA filament used to perform homology searching. When a terminus of the target DNA is complementary to the single-stranded portion of the oligonucleotide, ligation can covalently link the oligonucleotide, which can be labeled at the 5′ end with a detectable label, to the target DNA to allow detection or isolation of specific target DNA sequences without denaturation of the target DNA.

No applications of RecA have heretofore been proposed that allow the determination of repeat numbers in regions of tandem repeats.

### Recombinase Mediated Ligation (RML)

RML is a revolutionary method in that it:
1. Can utilize amplified, genomic or plasmid target DNA,
2. Does not require denaturation of target DNA,
3. Can allow extremely high order multiplexing,
4. Is compatible with a variety of assay formats and detection platforms, including microplates, microarrays, and microspheres combined with flow cytometry,
5. Is simple, low cost and robust,

Genotyping a two allele SNP via RML requires three oligonucleotides; a common oligonucleotide, one end of which terminates one base away from the SNP site, and two allele specific reporter oligonucleotides. The specificity of the reporter oligonucleotides is determined by the terminal base, which is complementary to one allele of the SNP. In the first step all 3 oligonucleotides are mixed with RecA in the presence of ATP, which allows the protein to bind to the single-stranded oligonucleotides to form RecA filaments. This reaction can be performed in the presence of double stranded DNA, thus target DNA can be added before or after RecA filament formation. RecA filaments engage in homology searching on double stranded DNA and form D-loops when regions of complementarity are encountered. When a common oligonucleotide and a reporter oligonucleotide complementary to the SNP allele in the target DNA are in a single D-loop such that the adjacent ends are properly base paired, the structure is a substrate for DNA ligase, which joins the common and reporter oligonucleotides to form an RML product. When the terminal nucleotide of the reporter oligonucleotide is not complementary to the target DNA sequence, ligation can not occur. RML requires ATP and is inhibited by ATP[gamma-S], which allows RecA filament formation and homology searching, but does not allow RecA dissociation from the filament, suggesting that it is necessary for RecA to be released from the filaments to allow ligation.

The ability of RML to genotype SNP sequences and to identify specific sequences does not in any way suggest that the process should be useful for determining the number of repeats in a tandem repeat sequence. Since it is known that large, non-homologous sequences at the termini of RML oligonucleotides opposite of the ligation sequences do not inhibit or limit RML, it might be expected that RML would allow ligation of any pair of oligonucleotides, one or both of which contain repeat sequences at their termini, i.e., formation of a ligation substrate should be expected to depend only on the sequence in the terminal portion of the oligonucleotides and presence of repeat sequences in said terminal portions should, therefore, allow multiple alignments with a region of tandem repeats.

### SUMMARY OF THE INVENTION

The present invention is directed to recombinase assisted methods for determining the number of repeat units in a tandem repeat containing region of double stranded DNA.

The RML method of tandem repeat genotyping includes the following steps:

(a) providing a single stranded DNA oligonucleotide probe (universal oligonucleotide) having known nucleotide sequence at least a portion of which is complementary to sequence on one side of a region of tandem repeats in the target DNA, which probe is optionally detectably labeled or contains an adduct to allow immobilization. The sequence of the probe may also contain one or more repeat sequence units.
(b) providing a set of single stranded DNA oligonucleotide probes (allele specific oligonucleotides) having known nucleotide sequence at least a portion of which is complementary to sequence on one side of a region of tandem repeats in the target DNA, the opposite side of that to which said universal oligonucleotide contains complementary sequence, which probe is optionally detectably labeled or contains an adduct to allow immobilization. The sequence of the probes also contain various numbers of repeat sequence units.
(c) contacting the probes with a RecA protein or a homologue thereof to form RecA filaments;
(d) contacting the RecA filaments with target double stranded DNA, thereby allowing homology searching by the RecA filaments and formation of a three stranded DNA D-loop structure in the target DNA, which D-loop structure comprises two oligonucleotides complementary to one of the strands of the target DNA and the two strands of the target DNA;
(e) contacting the D-loop structure with a DNA ligase under conditions that permit covalent bonding of directly adjacent ends of the correctly aligned oligonucleotides, i.e., a pair of oligonucleotides paired with one strand of the target such that there is no gap or overlap between the ends, to form linked oligonucleotide probe molecules; and
(f) detecting the linked oligonucleotide probe molecules and determining which of the set of allele specific oligonucleotides is found in the linked molecule, which allele specific oligonucleotide is diagnostic of the number of repeat units in the target DNA duplex.

The RML method of chimera detection comprises:

(a) providing a single stranded DNA oligonucleotide probe (universal oligonucleotide) having known nucleotide sequence at least a portion of which is complementary to sequence on one side of a region of tandem repeats in the target DNA, which probe is optionally detectably labeled or contains an adduct to allow immobilization. The sequence of the probe may also contain one or more repeat sequence units.
(b) providing a set of single stranded DNA oligonucleotide probes (allele specific oligonucleotides) having known nucleotide sequence at least a portion of which is complementary to sequence on one side of a region of tandem repeats in the target DNA, the opposite side of that to which said universal oligonucleotide contains complementary sequence, which probe is optionally detectably labeled or contains an adduct to allow immobilization. The sequence of the probes also contain various numbers of repeat sequence units.
(c) contacting the probes with a RecA protein or a homologue thereof to form RecA filaments;
(d) contacting the RecA filaments with target double stranded DNA, thereby allowing homology searching by the RecA filaments and formation of a three stranded DNA D-loop structure in the target DNA, which D-loop structure comprises two oligonucleotides complementary to one of the strands of the target DNA and the two strands of the target DNA;
(e) contacting the D-loop structure with a DNA ligase under conditions that permit covalent bonding of directly adjacent ends of the correctly aligned oligonucleotides, i.e., a pair of oligonucleotides paired with one strand of the target such that there is no gap or overlap between the ends, to form linked oligonucleotide probe molecules; and
(f) detecting the linked oligonucleotide probe molecules and determining which of the set of allele specific oligonucleotides are found in the linked molecule, wherein the presence of more than two allele specific oligonucleotides in linked molecules indicates that the target DNA sample is from a chimera.

In a particular embodiment, the RecA protein is from E. coli.

In the methods described herein, the labels may be any suitable detectable label, e.g., a fluorophore, a chromophore, a radionuclide, biotin, digoxigenin, etc. The probe DNAs, dNTPs or terminators may be directly labeled by direct bonding or binding of the label. However, the term “detectably labeled,” includes “indirect” labeling wherein the “detectable label” is a primary antibody, or any other binding partner, which is directly labeled. Alternatively, the detectable label may be a combination of an unlabeled primary antibody with a directly labeled secondary antibody specific for the primary antibody.

In the present method, probe DNA may be in solution or immobilized to any solid support and may be immobilized either before or after reaction with RecA and target DNA.

The present invention also provides a kit useful for practicing any of the above methods, the kit being adapted to receive therein one or more containers, the kit comprising:

(a) a first container containing RecA protein or a homologue thereof;
(b) a second container containing single stranded DNA oligonucleotides, which may optionally be detectably labeled or contain adducts to allow immobilization;
(c) a third container or plurality of containers containing buffers and reagent or reagents including a DNA ligase capable of linking DNA oligonucleotides when the oligonucleotides are annealed to target DNA without overlap or gap at their adjacent ends.

The kit may also include:

(a) a first container containing RecA protein or a homologue thereof,
(b) a second container containing single stranded DNA oligonucleotides, some of which may be immobilized to a solid support;
(c) a third container or plurality of containers containing buffers and reagent or reagents including additional DNA oligonucleotides and a DNA ligase capable of linking DNA probes when the probes are annealed to target DNA without a base pair mismatch at their adjacent ends.

### BRIEF DESCRIPTION OF THE DRAWINGS

FIG. 1 illustrates a schematic representation of RecA-mediated Ligation (RML) genotyping of the TPDX short tandem repeat (STR) region according to an embodiment of the present invention. A single “universal” oligonucleotide (blue) and multiple (10 in the example) “reporter” oligonucleotides are used to form RecA filaments, which in turn are used to perform RML (homology searching and ligation) on target DNA. Both the universal and reporter oligonucleotides are complementary to a portion of the sequence flanking the repeat region, in order to provide precise alignment of the oligonucleotides with the target DNA. Oligonucleotides also contain variable amounts of sequence complementary to the repeat portion of the target, i.e., variable numbers of repeats. One of the oligonucleotides may contain no repeats. Only that reporter oligonucleotide whose end anneals directly adjacent to the end of the universal oligonucleotide can be ligated. The specific reporter oligonucleotide which ligates to the universal oligonucleotide is diagnostic of the number of repeat units in the repeat region, i.e., the number of repeats is equal to the sum of the number of repeats in the universal oligonucleotide and the number in the successfully ligated reporter oligonucleotide.

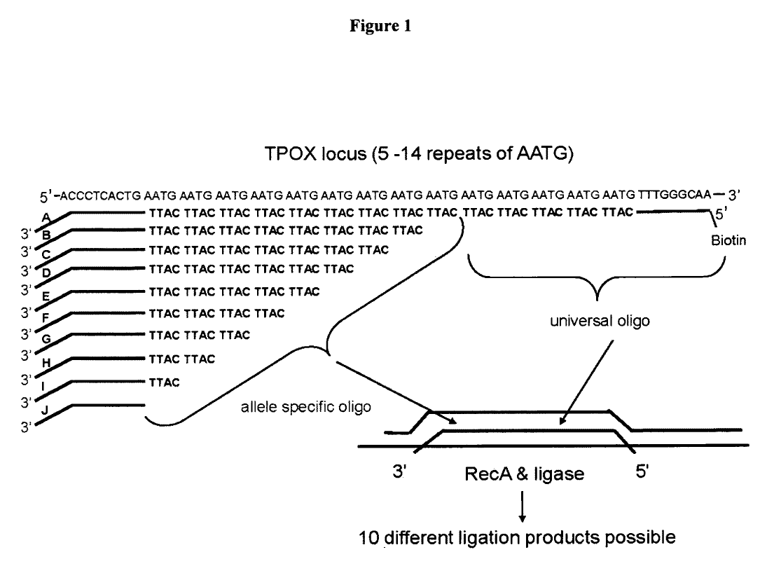

FIG. 2 illustrates data from an experiment using RML-STR genotyping to genotype TPDX STRs from four human DNA samples utilizing a microplate format according to an embodiment of the present invention. 4 nM each repeat specific reporter oligonucleotide, 40 nM common oligonucleotide, and 3.4 uM RecAE38K were incubated at 37° C. for 15 min to allow RecA filament formation. After addition of PCR amplified TPDX sequences (250-300 bp) from randomly selected human DNA samples and T4 Ligase, incubation was continued for 60 min before stopping the reaction with EDTA. The reaction was split into individual wells of a 96 well avidin coated microplate, and 1 pmole of one type of 5′ biotin labeled immobilization oligonucleotide was added to each of the wells to allow for hybridization and capture in a 30 min reaction at RT. The wells were washed before addition of Anti-Fitc AP conjugate (45 min at RT), followed by pNPP substrate. Absorbance was read at 405 nm after 30 min color development. Data are averages of duplicate reactions and are presented as the ratio of signal for a given oligonucleotide to background signal for that specific oligonucleotide (obtained in a control well in the same experiment).

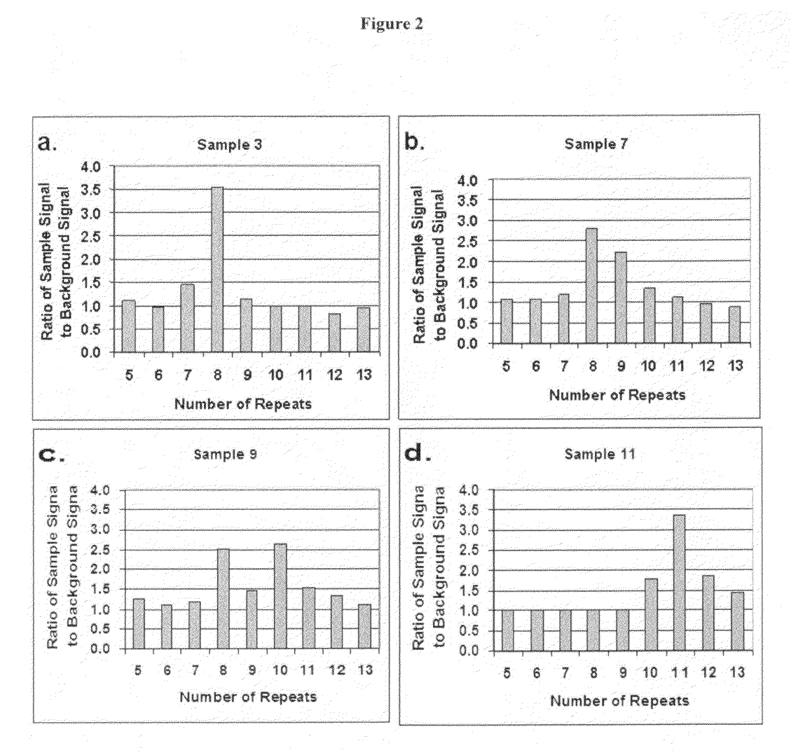

FIG. 3 illustrates RML-STR genotyping to genotype TPDX STRs from seven human DNA samples utilizing a microarray format according to an embodiment of the present invention. Allele-specific oligonucleotide mix (containing empirically optimized amount of each allele specific oligonucleotide between 4 nM and 12 nM), 40 nM universal oligonucleotide, and 3.4 uM RecAE38K were incubated at 37° C. for 15 min to allow RecA filament formation. After addition of PCR-amplified TPDX sequences and T4 ligase, incubation was continued for 30 min before stopping the reaction with EDTA. Reaction was diluted 1:2 by volume with 1×TBST+0.5% casein. Reaction volume was added to subarray and incubated at 50° C. for 30 min to allow hybridization to zip code capture oligos immobilized to microarray surface. Following 1×TBST wash, streptavidin-HRP (2 ug/ml) was added and incubated at RT for 10 min. Slide was washed and tyramide-Cy3 added (1:50 in amplification diluent) and incubation continued at RT for 10 min. Slide was washed, centrifuged to dry (600 RPM for 3 min), and scanned for Cy3 fluorescence. Signal is average of duplicate array spots measured for median signal intensity minus background.

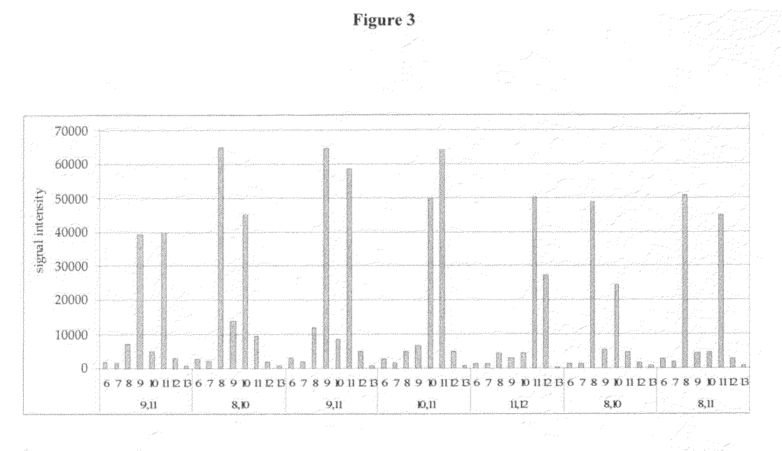

### DETAILED DESCRIPTION OF THE INVENTION

The present inventors have devised a novel technology for determining the number of repeat units in a region of double stranded DNA including a tandem repeat region (RML-STR) and determining if a given DNA sample is from a chimeric organism, based on RecA mediated homology searching followed by repeat number specific oligonucleotide ligation. In general, the present methods employ:

(1) a double stranded target or test DNA molecule, which may be any synthetic, viral, plasmid, prokaryotic or eukaryotic DNA from any source, including, but not limited to, genomic DNA, restriction digestion fragments or DNA amplified by PCR or by any other means;
(2) single stranded DNA oligonucleotide probes, which might be any synthetic oligonucleotide, PCR amplicon, plasmid DNA, viral DNA, bacterial DNA or any other DNA of known sequence or of sequence complementary to the target DNA or to a portion thereof,
(3) E. coli RecA or a homologue thereof, as defined below.

As used herein and in the present claims (for the sake of brevity and clarity), “RecA” is intended to include either the native or mutant E. coli RecA protein, or a “homologue” thereof as defined below. A “homologue” of a given protein is a protein that has functional and, preferably, also structural similarity to its “reference” protein. One type of homologue is encoded by a homologous gene from another species of the same genus or even from other genera. As described below, RecA-like proteins, originally discovered in bacteria, have eukaryotic homologues in groups ranging from yeast to mammals. A functional homologue must possess the biochemical and biological activity of its reference protein, particularly the DNA binding selectivity or specificity so that it has the utility described herein. In view of this functional characterization, use of homologues of E. coli RecA proteins, including proteins not yet discovered, fall within the scope of the invention if these proteins have sequence similarity and the described DNA binding or biological activity or “improved” binding activity. Non-limiting examples of improvements include a RecA homologue that binds to shorter DNA molecules or has a higher binding affinity for single stranded DNA.

“Homologues” is also intended to include those proteins which have been altered by mutagenesis or recombination that have been performed to improve the protein's desired function. These approaches are generally well described and well referenced below. Mutagenesis of a protein gene, conventional in the art, can be accomplished in vivo by cloning the gene into bacterial vectors and duplicating it in cells under mutagenic conditions, e.g., in the presence of mutagenic nucleotide analogs and/or under conditions in which mismatch repair is deficient. Mutagenesis in vitro, also well-known in the art, generally employs error-prone PCR wherein the desired gene is amplified under conditions (nucleotide analogues, biased triphosphate pools, etc.) that favor misincorporation by the PCR polymerase. PCR products are then cloned into expression vectors and the resulting proteins examined for function in bacterial cells.

Recombination generally involves mixing homologous genes from different species, allowing them to recombine, frequently under mutagenic conditions, and selecting or screening for improved function of the proteins from the recombined genes. This recombination may be accomplished in vivo, most commonly in bacterial cells under mismatch repair-deficient conditions which allow recombination between diverged sequences and also increase the generation of mutations. In addition, Stemmer and colleagues have devised methods for both in vivo and in vitro recombination of diverged sequences to create “improved” proteins. Most involve PCR “shuffling” wherein two PCR amplicons of diverged sequences are digested and mixed together such that the fragments serve as both primer and template for additional PCR and, in so doing, combine different segments of the diverged genes, which is, in effect, genetic “recombination.” Frequently, error prone PCR conditions are included to further stimulate generation of novel sequences. Resulting PCR products are cloned into expression vectors, and the resulting proteins are screened for improved function. Such methods are well known to those skilled in the art.

As noted, homologues of the present invention generally share sequence similarity with their reference protein. To determine the % identity of two amino acid sequences (or of two nucleic acid sequences), the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred method of alignment, Cys residues are aligned. The length of a sequence being compared is at least 30%, preferably at least 40%, more preferably at least 50%, even more preferably at least 60%, and even more preferably at least 70%, 80%, or 90% of the length of the reference sequence (e.g., E. coli RecA). The amino acid residues (or nucleotides) at corresponding amino acid (or nucleotide) positions are then compared. When a position in the first sequence is occupied by the same amino acid residue (or nucleotide) as the corresponding position in the second sequence, then the molecules are identical at that position. As used herein amino acid or nucleic acid “identity” is also to be considered amino acid or nucleic acid “homology”. The percent identity between the two sequences is a function of the number of identical positions shared by the sequences, taking into account the number of gaps and the length of each gap which need to be introduced for optimal alignment. The comparison of sequences and determination of percent identity between two sequences can be accomplished using mathematical algorithms, e.g., the Needleman and Wunsch (J. Mol. Biol. 48:444-453 (1970)) algorithm which has been incorporated into the GAP program (see below) using either a Blossom 62 matrix or a PAM250 matrix. A preferred program, “GAP” in the GCG software package, available at http://www.gcg.com, uses a NWSgapdna.CMP matrix and a gap weight of 40, 50, 60, 70, or 80 and a length weight of 1, 2, 3, 4, 5, or 6. In another approach, the % identity between two amino acid or nucleotide sequences is determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4.

The nucleic acid or protein sequence of a particular RecA protein can further be used as a “query sequence” to perform a search against public databases, for example, to identify other family members or related sequences. Such searches can be performed using the NBLAST and XBLAST programs (version 2.0) of Altschul et al. (1990) J. Mol. Biol. 215:403-410. To obtain gapped alignments for comparison purposes, Gapped BLAST can be utilized as described in Altschul et al. (1997) Nucl. Acids Res. 25:3389-3402. When using BLAST and Gapped BLAST, the default parameters of the respective programs can be used. See http://www.ncbi.nlm.nih.gov. For example, BLAST nucleotide searches can be performed with the NBLAST program, score=100, wordlength=12 to obtain nucleotide sequences homologous to a query RecA or SSB coding nucleic acid sequence. BLAST protein searches can be performed with the XBLAST program, preferably set at score=50, wordlength=3, to obtain amino acid sequences homologous to a query RecA or SSB protein molecule (e.g., wild-type sequence from E. coli).

Thus, in a particular embodiment of the present invention, an exemplary homologue of an E. coli RecA protein protein has, first and foremost, (a) the functional activity of native E. coli RecA and also preferably shares (b) a sequence similarity to the native E. coli protein, when determined as above, of at least about 20% (at the amino acid level), preferably at least about 40%, more preferably at least about 60%, even more preferably at least about 70%, even more preferably at least about 80%, and even more preferably at least about 90%.

At least 65 RecA genes from different bacteria have been cloned and sequenced. Eukaryotic homologues of RecA have been identified in every eukaryotic species examined; the prototype eukaryotic RecA homologue is the yeast Rad51 protein. Therefore, any homologue of E. coli RecA which, like the E. coli protein, forms DNA filaments for initiation of genetic recombination as well as any functional form that has been mutated or evolved in vivo or in vitro is included within the scope of the present invention.

RecA functions in vitro, forming a three stranded structure involving oligonucleotides along sequence stretches as short as 15 nucleotides. Combining the activities of RecA with genotype-specific oligonucleotide ligation creates a most powerful system for determination of repeat numbers in regions of tandem repeats in which RecA-coated single strandedDNA catalyzes formation of a three strand (D-loop) structure without the need for prior denaturation of the test double stranded DNA.

In one particular embodiment, the present system employs: (1) RecA; (2) allele (i.e., repeat number) specific oligonucleotides that contain adducts to allow immobilization; and (3) universal oligonucleotides containing an internal or terminal detectable label which are ligation partners for the allele specific oligonucleotides.

Oligonucleotides are exposed to RecA under conditions that allow RecA filament formation. Filaments are exposed to double stranded target DNA under conditions the allow RecA-mediated homology searching, the formation of D-loops and ligation of properly aligned oligonucleotides, wherein ligation occurs only when the sum of the number of repeat units in the universal oligonucleotide and the particular allele specific oligonucleotide is equal to the number of repeat units in the target duplex in the D-loop.

Ligation may be accomplished by any DNA ligase, including, but not limited to, T4 ligase, E. coli ligase, Taq ligase, Rtth ligase and the like.

The ligation partner oligonucleotides may be of any length but are preferably synthetic oligonucleotides, of about 60-90 bases in length, are specific for a region in double stranded DNA sample that contains tandem repeats. All oligonucleotides must contain at least some sequence complementary to non-repeat sequence flanking the region of repeats.

The target DNA may be of any length (up to an entire chromosome) and can be either genomic or plasmid DNA or a PCR amplicon.

The detectably-labeled oligonucleotides can be directly labeled with fluorophores or fluorescent labels, including, but not limited to, fluorescein (and derivatives), 6-Fam, Hex, tetramethylrhodamine, cyanine-5, CY-3, allophycocyanin, Lucifer yellow CF, Texas Red, rhodamine, Tamra, Rox, Dabcyl, etc. They may also be labeled with radioactive labels, digoxigenin, chemiluminescent labels or colorimetric labels.

RecA filament formation can be accomplished, for example, in a Tris-HCl or Tris-acetate buffer, (20-40 mM, pH 7.4-7.9) with MgCl.sub.2 or Mg acetate (1-4 mM), dithiothreitol (0.2-0.5 mM), and ATP or ATP[.gamma.-S] (0.3-1.5 mM). If ATP is used, an ATP regenerating system comprising phosphocreatine and creatine kinase may be included. RecA and oligonucleotide are generally added at a molar ratio of 0.1-3 (RecA:nucleotides). Incubation is at room temperature or, preferably, 37.degree. C., for 5-30 min. D-loop or triple strand structure formation involves adding RecA filaments to double stranded DNA and incubating, preferably at 37.degree. C., for about 15 min-2 hrs. It is also possible to form RecA filaments and do homology searching in a single reaction vessel, i.e., to mix RecA with oligonucleotides and double stranded DNA at the same time.

When immobilization oligonucleotides are employed, immobilization may be to any suitable surface such as a microtiter plate, magnetic beads, beads suitable for detection via flow cytometry, etc. Immobilization may be direct to the solid support or may occur via hybridization of the allele specific oligonucleotides to other oligonucleotides immobilized to a solid support.

In one particular embodiment of this invention immobilization may occur following ligation by hybridization of allele specific oligonucleotides to oligonucleotides immobilized to separate wells of a microtiter plate or separate spots of a microarray slide or flow chamber. In this case, each allele specific oligonucleotide contains a different sequence (eg., zip code sequence) at one end (i.e., the end distal to the repeat sequence) that is not complementary to the target DNA and that allows specific immobilization of a allele specific oligonucleotide by specific hybridization to immobilization oligonucleotides immobilized to individual wells of a microtiter plate, which immobilization may occur before or after hybridization to the allele specific oligonucleotide. If a given allele specific oligonucleotide has been ligated to the universal oligonucleotide, i.e., if the DNA sample contains a repeat region with a repeat number equal to the sum of the number of repeat units in the universal oligonucleotide and the particular allele specific oligonucleotide, label will be detected in that well. Samples homozygous for a particular repeat number will give signal in only a single well. Heterozygous samples will show signal in two wells. Signal in more than two wells indicate that the sample is a chimera. Sample data from RML-STR genotyping of human DNA samples via microtiter plate

In another particular embodiment of this invention allele specific oligonucleotides are immobilized to a solid support prior to RecA filament formation and homology searching. In this case, allele specific oligonucleotides are kept separate by, for example, being in different spots on a microarray slide or by being on separate beads, thus allowing high order multiplexing, i.e., simultaneously genotyping multiple STR regions from a single sample. For example, microarray based genotyping of an STR region with 10 different alleles (10 repeat number variants) will require 10 spots, but only a single, labeled universal oligonucleotide. Oligonucleotides may be immobilized via means well know in the art, including, but not limited to, amine-aldehyde coupling or biotin-streptavidin binding. Label will be detected in one spot for each genotype present in the test sample. Homozygous samples will have signal present in only a single spot and heterozygous samples will have signals in two spots. Signals in more than two spots indicate that the sample is chimeric.

Detection of label may be accomplished by a variety of methods including, but not limited to, plate readers capable of detecting visible light or fluorescent signals, use of microchip and microarray readers and flow cytometry.

Labeling can be of any of the type mentioned above and may be restricted only to the universal oligonucleotides. Similarly, the oligonucleotides can be modified by any method to allow immobilization.

A major advantage of the RecA-mediated ligation based method of STR genotyping is that it can operate on genomic DNA without denaturation or amplification. In addition, the method allows precise determination of repeat length and does so by precisely aligning ligation partner oligonucleotides based on unique sequence flanking the repeat region.

It is difficult to overstate the power of the RML-STR method. It is rapid, works with small samples and can readily be adapted to clinical applications for forensic or diagnostic genotyping.

### Kits

The present invention is also directed to kit or reagent systems useful for practicing the methods described herein. Such kits will contain a reagent combination including the elements required to conduct an assay according to the methods disclosed herein. The reagent system is presented in a commercially packaged form, as a composition or admixture where the compatibility of the reagents will allow, in a test device configuration, or more typically as a test kit, i.e., a packaged combination of one or more containers, devices, or the like holding the necessary reagents, and preferably including written instructions for the performance of assays. The kit of the present invention may be adapted for any configuration of assay and may include compositions for performing any of the various assay formats described herein.

Kits containing RecA, oligonucleotides and, where applicable, reagents for detection of fluorescent, chemiluminescent, radioactive or colorimetric signals, are within the scope of this invention. In one embodiment, a kit of this invention designed to allow determination of repeat unit numbers in specific sequences of target DNA, includes one universal oligonucleotide probe and a set of allele specific oligonucleotides specific for a selected region of tandem repeats. The oligonucleotides may be labeled as described above. The kits also include a plurality of containers of appropriate buffers and reagents.

The references cited above are all incorporated by reference herein, whether specifically incorporated or not.

Having now fully described this invention, it will be appreciated by those skilled in the art that the same can be performed within a wide range of equivalent parameters, concentrations, and conditions without departing from the spirit and scope of the invention and without undue experimentation.

What is claimed is:

1. A method of determining the number of repeat units in double stranded DNA comprising:
a. a first single stranded DNA oligonucleotide or set of oligonucleotides complementary to a specific region of target DNA wherein the 3′ end of said first oligonucleotide or the 3′ end of each member of said oligonucleotide set is complementary to a region of said target DNA 3′ to a region of repeat units and wherein the 3′ end of said first oligonucleotide or the 3′ end of each member of said oligonucleotide set is complementary to a region of said target DNA 5′ to a region of repeat units and wherein said first oligonucleotide or each member of said oligonucleotide set contains one or more repeat units at the 5′ end;
b. a second single stranded DNA oligonucleotide or set of oligonucleotides complementary to a specific region of target DNA wherein the 5′ end of said second oligonucleotide or the 5′ end of each member of said second oligonucleotide set is complementary to a region of said target DNA 5′ to a region of repeat units and wherein said oligonucleotide or set of oligonucleotides contains one or more repeat units at its 3′ end;
c. contacting said first and second oligonucleotides with RecA protein or a homologue of RecA from any species to form RecA filaments;
d. contacting said target DNA with said RecA filaments to form duplex DNA between said oligonucleotides and one strand of said target DNA;
e. ligation of said oligonucleotides by DNA ligase under conditions wherein ligation is dependent upon the 5′ end of said first oligonucleotide and the 3′ end of said second oligonucleotide being based paired with adjacent bases in said target DNA strand; and
f. detection of ligation product wherein formation of said ligation product reveals said number of repeat units in said region of repeat units in said target DNA.

2. The method of claim 1 wherein said first or second oligonucleotide is labeled with a label selected from the group consisting of fluorescent, radioactive, chemiluminescent, enzymatic, antigenic and colorimetric.

3. The method of claim 1 wherein said first or second oligonucleotide further comprises an adduct that allows immobilization of the oligonucleotide to a solid support before or after said RecA filament formation or ligation.

4. The method of claim 3 wherein said adduct is selected from the group consisting of an oligonucleotide and an adduct.

5. The method of claim 4, wherein said adduct is selected from the group consisting of biotin and digoxigenin.

6. The method of claim 1, wherein said RecA is from E. coli.

7. A method for detecting chimeric cells or organisms comprising:
a. a first single stranded DNA oligonucleotide or set of oligonucleotides complementary to a specific region of target DNA in the genome of said organism or cell wherein the 3′ end of said first oligonucleotide or the 3′ end of each member of said oligonucleotide set is complementary to a region of said target DNA 5′ to a region of repeat units and wherein said first oligonucleotide or each member of said oligonucleotide set contains one or more repeat units at the 5′ end;
b. a second single stranded DNA oligonucleotide set of three or more oligonucleotides complementary to a specific region of said target DNA wherein the 5′ end of each member of said second oligonucleotide set is complementary to a region of said target DNA 5′ to a region of repeat units and wherein said oligonucleotide or oligonucleotide set contains one or more repeat units at its 3′ end;
c. contacting said first and second oligonucleotides with RecA protein or a homolog of RecA from any species to form RecA filaments;
d. contacting said target DNA with said RecA filaments to form duplex DNA between said oligonucleotides and one strand of said target DNA;
e. ligation of said oligonucleotides by DNA ligase under conditions wherein ligation is dependent upon the 5′ end of said first oligonucleotide and the 3′ end of said second oligonucleotide being based paired with adjacent bases in said target DNA strand; and
f. detection of ligation products wherein formation of said ligation product reveals number of repeat units in said region of repeat units in said target DNA and wherein detection of more than two different ligation products from a single cell or organism is diagnostic of that cell or organism being chimeric.

8. The method of claim 7, wherein said first or second oligonucleotide is labeled with a label selected from the group consisting of fluorescent, radioactive, chemiluminescent, enzymatic, antigenic and colorimetric.

9. The method of claim 7, wherein said first or second oligonucleotide further comprises an adduct that allows immobilization of the oligonucleotide before or after said RecA filament formation or ligation.

10. The method of claim 9, wherein said adduct is selected from the group consisting of an oligonucleotide, an amine group, biotin and digoxigenin.

11. The method of claim 7 wherein the RecA is from E. coli.


SEQUENCE LISTING (12 sequences)

SEQ ID NO: 1 - DNA, 75 bp, Artificial Sequence
  Artificial sequence based on human TPOX short tandem repeat (STR) region utilized as “universal” oligonucleotide per Figure 1. Sequence 5′ to 3′.
accctcactgaatgaatgaatgaatgaatgaatgaatgaatgaatgaatgaatgaatgaatgaatgtttgggcaa

SEQ ID NO: 2 - DNA, 29 bp, Artificial Sequence
  Artificial sequence based on human TPOX short tandem repeat (STR) region utilized as one of many “reporter” oligonucleotides utilized to form RecA filaments per Figure 1. Sequence 3′ to 5′.
ttgcccaaacattcattcattcattcatt

SEQ ID NO: 3 - DNA, 46 bp, Artificial Sequence
  Artificial sequence based on human TPOX short tandem repeat (STR) region utilized as one of many “reporter” oligonucleotides utilized to form RecA filaments per Figure 1. Sequence 3′ to 5′.
tgggagtgacttacttacttacttacttacttacttacttacttac

SEQ ID NO: 4 - DNA, 42 bp, Artificial Sequence
  Artificial sequence based on human TPOX short tandem repeat (STR) region utilized as one of many “reporter” oligonucleotides utilized to form RecA filaments per Figure 1. Sequence 3′ to 5′.
tgggagtgacttacttacttacttacttacttacttacttac

SEQ ID NO: 5 - DNA, 38 bp, Artificial Sequence
  Artificial sequence based on human TPOX short tandem repeat (STR) region utilized as one of many “reporter” oligonucleotides utilized to form RecA filaments per Figure 1. Sequence 3′ to 5′.
tgggagtgacttacttacttacttacttacttacttac

SEQ ID NO: 6 - DNA, 34 bp, Artificial Sequence
  Artificial sequence based on human TPOX short tandem repeat (STR) region utilized as one of many “reporter” oligonucleotides utilized to form RecA filaments per Figure 1. Sequence 3′ to 5′.
tgggagtgacttacttacttacttacttacttac

SEQ ID NO: 7 - DNA, 30 bp, Artificial Sequence
  Artificial sequence based on human TPOX short tandem repeat (STR) region utilized as one of many “reporter” oligonucleotides utilized to form RecA filaments per Figure 1. Sequence 3′ to 5′.
tgggagtgacttacttacttacttacttac

SEQ ID NO: 8 - DNA, 26 bp, Artificial Sequence
  Artificial sequence based on human TPOX short tandem repeat (STR) region utilized as one of many “reporter” oligonucleotides utilized to form RecA filaments per Figure 1. Sequence 3′ to 5′.
tgggagtgacttacttacttacttac

SEQ ID NO: 9 - DNA, 22 bp, Artificial Sequence
  Artificial sequence based on human TPOX short tandem repeat (STR) region utilized as one of many “reporter” oligonucleotides utilized to form RecA filaments per Figure 1. Sequence 3′ to 5′.
tgggagtgacttacttacttac

SEQ ID NO: 10 - DNA, 18 bp, Artificial Sequence
  Artificial sequence based on human TPOX short tandem repeat (STR) region utilized as one of many “reporter” oligonucleotides utilized to form RecA filaments per Figure 1. Sequence 3′ to 5′.
tgggagtgacttacttac

SEQ ID NO: 11 - DNA, 14 bp, Artificial Sequence
  Artificial sequence based on human TPOX short tandem repeat (STR) region utilized as one of many “reporter” oligonucleotides utilized to form RecA filaments per Figure 1. Sequence 3′ to 5′.
tgggagtgacttac

SEQ ID NO: 12 - DNA, 10 bp, Artificial Sequence
  Artificial sequence based on human TPOX short tandem repeat (STR) region utilized as one of many “reporter” oligonucleotides utilized to form RecA filaments per Figure 1. Sequence 3′ to 5′.
tgggagtgac